(19) United States
(12) Patent Application Publication
(10) Pub. No.: US 2025/0281686 A1
(43) Pub. Date: Sep. 11, 2025

(54) DELIVERY OF MEDICINAL GAS IN A LIQUID MEDIUM

(71) Applicant: Third Pole, Inc., Waltham, MA (US)

(72) Inventors: Xiaokun Dong, Medford, MA (US); Wolfgang Scholz, Beverly, MA (US); Gregory W. Hall, Belmont, MA (US); Basil Athenson, Lake Forest, IL (US)

(73) Assignee: Third Pole, Inc., Waltham, MA (US)

(21) Appl. No.: 18/925,929

(22) Filed: Oct. 24, 2024

Related U.S. Application Data
(60) Provisional application No. 63/592,778, filed on Oct. 24, 2023.
(60) Provisional application No. 63/602,902, filed on Nov. 27, 2023.

(51) Int. Cl. A61M 5/14; A61K 33/00; A61K 47/22; A61M 5/00

(52) U.S. Cl. CPC A61M 5/1407; A61K 33/00; A61K 47/22; A61M 2005/006; A61M 2202/0275

(57) ABSTRACT

Systems and methods for delivering a gas, such as nitric oxide, are provided. In some embodiments, systems and methods are provided for delivering a gas, such as nitric oxide, in the form of nanobubbles and/or a dissolved gas using a liquid medium.

### RELATED APPLICATIONS

This application claims the benefit of and priority to U.S. Provisional Application No. 63/602,902 filed Nov. 27, 2023, and U.S. Provisional Application No. 63/592,778 filed Oct. 24, 2023, and the contents of each of these applications are hereby incorporated herein by reference in their entireties,

### FIELD

The present disclosure relates to systems and methods for delivering medicinal gas using a liquid medium.

### SUMMARY

The present disclosure is directed to systems and methods for delivering a gas in the form of nanobubbles and/or a dissolved gas using a liquid medium. In some embodiments, systems and methods involving the delivery of nitric oxide to enhance uptake and/or delivery of other drugs are presented.

In some embodiments, a system is provided for generating a medicinal solution, including a reservoir configured to store a liquid a source of medicinal gas, a pump configured to propel the liquid from the reservoir, a nanobubble generator in fluid communication with the reservoir and the source of medicinal gas, the nanobubble generator configured to form a nanobubble solution including nanobubbles of the medicinal gas in the liquid, and a source of a reducing agent configured to be added to nanobubble solution to reduce nitrite to NO and dehydroascorbic acid in the nanobubble solution.

In some embodiments, the reducing agent is ascorbic acid, Vitamin E, or potassium iodide.

In some embodiments, a nanobubble solution is provided, including a liquid medium, a medicinal gas in the form of nitric oxide (NO), and a reducing agent configured to reduce nitrite to NO and dehydroascorbic acid in the nanobubble solution.

In some embodiments, the nanobubble solution is used to treat vasospasm, subarachnoid hemorrhage, Raynaud's disease, sexual disfunction, infections, or frostbite.

In some embodiment, the solution further includes an alkaline agent to neutralize the nanobubble solution before administration to a patient.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1A depicts an embodiment of a nanobubble generation system;

FIG. 1B depicts an embodiment of a nanobubble generation system;

FIG. 2 depicts an embodiment of a nanobubble generation system;

FIG. 3 depicts exemplary inputs and outputs for a nanobubble generation system controller;

FIG. 4 depicts an exemplary embodiment of an oxygen nanobubble generation system that utilizes an oxygen concentrator to source oxygen;

FIG. 5 illustrates an embodiment of a balloon catheter configured to deliver a NO nanobubble solution;

FIG. 6 depicts an exemplary embodiment of a system for delivering NO nanobubbles through the skin using a pressure gradient;

FIG. 7 depicts an exemplary embodiment of a nanobubble generation system with sparge gas flow;

FIG. 8 depicts an exemplary operation flow chart for a nanobubble generation system;

FIG. 9 depicts an exemplary embodiment of a reducing scrubber with optional filter;

FIG. 10 depicts an exemplary embodiment of a NO nanobubble generation system with an inline reducing scrubber;

FIG. 11A depicts an exemplary embodiment of a NO nanobubble generation system with a reducing scrubber in the drain line;

FIG. 11B depicts an exemplary embodiment of a system for passing a nanobubble solution through a reducing scrubber as a container is filled;

FIG. 11C depicts an exemplary embodiment of a system for passing a nanobubble solution within a syringe through a reducing scrubber;

FIG. 12 depicts an exemplary embodiment of a dual-reservoir syringe with mixing chamber for neutralizing a NO solution;

FIG. 13A depicts an exemplary embodiment of a dual-reservoir system that mixes and neutralizes as it drains to a patient;

FIG. 13B depicts an exemplary embodiment of a dual reservoir system that enables loading a single syringe with two solutions;

FIG. 14 depicts an exemplary embodiment of an open-top container with NO solution and inert gas headspace neutralized by a solid base;

FIG. 15 depicts an exemplary embodiment of an anaerobic nanobubble generation process flow;

FIG. 16 illustrates an exemplary embodiment of a packaging scheme for a NO solution;

FIG. 17 depicts another exemplary embodiment of a packaging scheme for a NO solution; and

FIG. 18 depicts another exemplary embodiment of a packaging scheme for a NO solution.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments.

Specific details are given in the following description to provide a thorough understanding of the embodiments. However, it will be understood by one of ordinary skill in the art that the embodiments may be practiced without these specific details. For example, systems, processes, and other elements in the presently disclosed embodiments may be shown as components in block diagram form in order not to obscure the embodiments in unnecessary detail. In other instances, well-known processes, structures, and techniques may be shown without unnecessary detail in order to avoid obscuring the embodiments. Figures depicting architectures forgo the details of also depicting cabling and control elements to provide focus on the innovation.

Subject matter will now be described more fully with reference to the accompanying drawings, which form a part hereof, and which show, by way of illustration, specific example aspects and embodiments of the present disclosure. Subject matter may, however, be embodied in a variety of different forms and, therefore, covered or claimed subject matter is intended to be construed as not being limited to any example embodiments set forth herein; example embodiments are provided merely to be illustrative. The following detailed description is, therefore, not intended to be taken in a limiting sense.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure relates to systems and methods for delivery of NO, oxygen or other gases contained within a liquid carrier instead of a gaseous carrier. This approach can provide purity, dose control, and gas conservation.

Nanobubbles are defined as bubbles with a diameter of <500 nm. These bubbles are small enough to enter and interact with the internal components of living cells. In some embodiments, the nanobubbles have a diameter of <200 nm or even <100 nm.

Nanobubbles are different than microbubbles. Nanobubbles can remain stable within a liquid for extended periods of time (e.g., weeks or more). Microbubbles float to the surface of a liquid. Nanobubble size and zeta potential (i.e., the potential difference between the molecules at the surface of the bubble and the liquid medium) are indicators of how stable the nanobubbles will be over time. Microbubbles tend to coalesce and turn into larger bubbles. Nanobubbles are so small that they electrically repel each other. As some nanobubble solutions age, however, their nanobubbles eventually coalesce to create larger bubbles. In other nanobubble solutions, high pressure gas within the nanobubbles escapes to surrounding liquid by dissolving. This dissolving continues until the pressure within the nanobubble has been exhausted at which time gas content within the liquid will be solely determined by gas conditions (i.e. pressure, temperature, gas mixture type) above the meniscus of the liquid.

A typical human cell has a diameter of around 100 microns. At a size of <200 nm, a nanobubble is small enough to enter living cells through the cell membrane and interact with a cell's organelles (i.e., the internal cellular components).

A nanobubble solution can vary in physical properties, depending on the application. For example, the solution may be a liquid, a gel, a hydrogel, an organogel, an emulsion, a microemulsion, a bigel, or an emulgel (for delivery of hydrophobic substances). A gas (e.g., nitric oxide, oxygen, etc.) can be utilized to make nanobubbles within a liquid. The liquid can be aqueous, lipid (e.g., oil, such as mineral oil), alcohol or other chemistry. Examples of oils include sesame oil, silicone oil, and mineral oil. Alcohol (e.g., ethanol, isopropyl alcohol) can be used in some applications because it can evaporate, rather than accumulate. Other liquids that are suitable for nanobubble solutions include, heparin, saline, heparinized saline, lactated Ringer's solution, phosphate-buffered saline, and biological fluids (e.g., plasma). In some embodiments, the liquid of a nanobubble solution consists of a liquid drug such as cough syrup, lavage solution, dialysis solution, enema solution, throat spray, contrast solution (MRI, Ultrasound, X-Ray (e.g., iodinated compounds), antibiotic solution. In some embodiments, the liquid can be a perfluorocarbon. In some embodiments, the liquid can include an emulsifier to assist the suspension of the bubbles after formation. Examples of materials that are added to either thicken or emulsify a nanobubble solution include hydrocolloids, gums (guar, gellan, acacia, xanthum), glycerin, sugar, starch (e.g., potato, corn), aloe, dextran, dextrin, maltodextrin, 5-10% dextrose solution, povidone (dissolves in water and oil), agar, pregel (modified starch), pectin, polylactic acid (PLA), polylactic-co-glycolic acid (PLGA), polyethylene glycol (PEG), cellulose, gelatin, collagen, chitosan, and alginate, acetic acid esters, ammonium phosphatide, glycan, brominated vegetable oil, cellulose, carboxymethylcellulose, diacetyl tartaric acid ester, dextrin, lactic acid esters, carrageenan, products from algae (e.g. algin, agar), lecithin (e.g. soy or egg based), diacetyl tartaric acid ester of monoglyceride, mustard, sodium stearoyl lactylate, magnesium stearate, mono and diglycerides, phosphates, polyglycerol esters, polysorbate (e.g. 60, 65, 80), propylene glycol esters of fatty acids, sodium stearolyl-lactylate, sorbitan monostearate, sucrose acetate isobutyrate, sucrose fatty acid ester, and sodium phosphates.

Nitric oxide gas within the nanobubbles can be 100% pure or diluted with an inert gas (e.g., nitrogen, helium, argon, carbon dioxide). It should be understood that nanobubbles can be made from other gases (e.g., carbon monoxide, carbon dioxide, ozone, oxygen, helium, nitrogen, nitrous oxide, and xenon), depending on the desired physiological effect.

Non-soluble anesthetic gases can also be utilized to form nanobubbles in a solution. Current IV-administered anesthetics are not ideal. For example, fentanyl is not absorbed by tissue well, and propofol can absorb well but is injected in a lipid medium which can result in excessive lipid in the blood stream. Most inhaled anesthetic substances are volatile delivered as a vapor (not a gas) because they are a liquid at room temperature. Ideal anesthetic gases for nanobubble delivery are gaseous at body temperature (e.g. ether, diethyl ether, cyclopropane, and desflurane).

The highest density of nanobubbles in an aqueous solution are found with gases that have a high dipole moment (e.g. ether, nitric oxide).

Various general techniques can be employed, alone or in combination, to form nanobubbles. In some embodiments, gas is flowed through a porous material to form columns of gas. A flow of fluid shears off the columns of gas into bubbles. In some embodiments, the porous material is a tube. In some embodiments, the porous material is a planar material (e.g., a membrane). In some embodiments, the porous material is in the shape of a tube or cylinder. In some embodiments, one or more columns of gas flow are introduced to a fluid vessel, and a stirring mechanism can create circular flow in the liquid that shears off the gas columns into bubbles. In some embodiments, a venturi can be used, such that liquid flows through a conduit, and as a diameter of the conduit narrows, the liquid velocity increases. The fast-flowing liquid velocity induces a low pressure at the wall of the conduit. Gas is drawn in through one or more orifices in the wall of the venturi and is sheared into nanobubbles. In some embodiments, laminar liquid flow through the venturi is used. In some embodiments, gas is introduced through multiple orifices around the periphery of the venturi. This can improve gas mixing and introduction to the liquid while supporting the use of a larger diameter venturi for laminar flow.

In some embodiments, ultrasound can be used. For example, an ultrasonic actuator in contact with a liquid can induce cavitation in the liquid. The cavities within the liquid can fill with dissolved gas to form nanobubbles. In some embodiments, a pressurized vessel can be used. For example, a vessel can be partially filled with a liquid, and a reservoir with one or more tiny holes is placed within the vessel along with pressurized gas in the headspace. The vessel is sealed until the time of use. Pressurized gas and liquid enter the reservoir to balance the high pressure inside and outside of the reservoir. Prior to use, a valve in the vessel wall is opened expose the contents to a lower pressure (typically atmospheric pressure). Gas within the headspace immediately escapes the vessel. Gas within the sphere escapes the sphere through the tiny holes, making nanobubbles within the liquid.

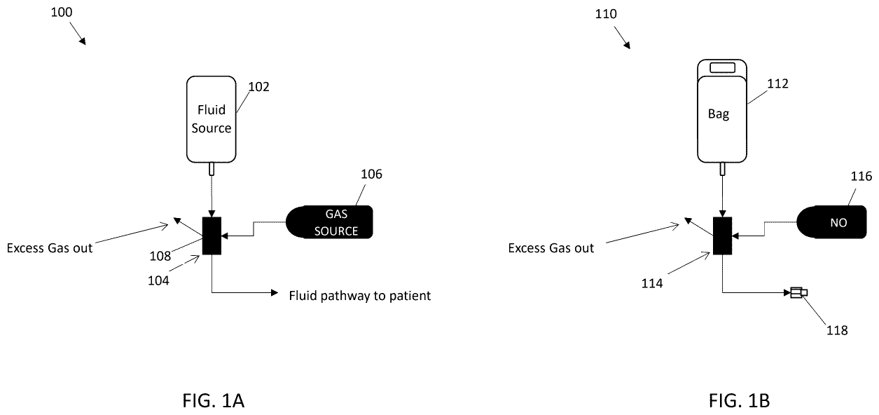

Various system architectures can be used to generate and/or deliver nanobubbles to a patient to treat a variety of medical conditions. In some embodiments, as shown in FIG. 1A, a nanobubble system 100 can include a liquid that can be sourced from a reservoir 102. A nanobubble generator 104 is configured to receive the liquid from the reservoir 102 as well as a gas from a gas source 106 such that the nanobubble generator 104 forms a plurality of nanobubbles from the gas in the liquid. Any excess gas is released from the nanobubble generator 104 through an outlet 108, such as a valve or other opening in the nanobubble generator. The nanobubbles are delivered to a patient through tubing, a cannula, catheter, a needle, or other delivery device.

FIG. 1B depicts an exemplary embodiment of a bedside NO nanobubble generation system 110. Liquid is sourced from a reservoir 112 (e.g., a bag, such as an IV bag) and passes into a nanobubble generator 114. NO gas from an NO source 116 enters the nanobubble generator 114 as well. Excess gas (i.e. gas that is not formed into nanobubbles) is released to the environment or to house vacuum. Liquid containing nanobubbles exits out of the nanobubble generator 114 and flows to an exit point. In the depicted example, the exit point is in the form of a connector 118, such as a Luer fitting that connects to a catheter. In some embodiments, a system like this provides nanobubble solution for intravascular delivery. In some embodiments, various levels of pressure and flow control for the liquid and gas (not shown) are applied to balance the delivery of solution constituents and/or target a particular solution flow rate and gas concentration within the liquid. For example, one of the means to vary the delivered dose of gaseous drug is to vary the concentration of the gas in the nanobubbles, such as using pure NO or NO diluted with nitrogen as the gas source. In some embodiments, there can be a plurality of gas reservoirs. A controller can be used to blend an active gas (e.g., NO) and an inert gas (e.g., nitrogen) to a specific concentration before introducing the gas to the nanobubble generator.

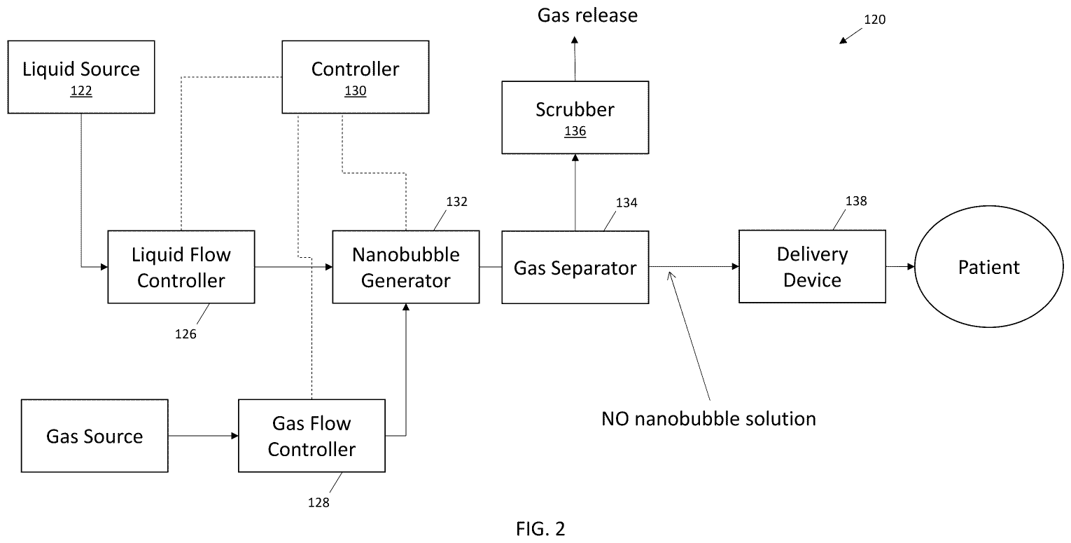

FIG. 2 depicts another exemplary embodiment of a nanobubble generation system 120. Liquid and gas are sourced from respective sources 122, 124 and flow through respective flow controllers 126, 128. A controller 130 modulates the flow of liquid and gas through their respective flow controllers 126, 128. Examples of flow controllers include but are not limited to pumps, valves, pressure regulators, and orifices. The gas and liquid enter a nanobubble generator 132 where nanobubbles of the gas are formed within the liquid. In some embodiments, the gas and liquid flows are merged before the nanobubble generator (not shown). In some embodiments, the nanobubble generation technology is controlled by the controller (shown). In some embodiments, the nanobubble generator is a passive device requiring no external control. The nanobubble formation process is not typically 100% efficient, leaving microbubbles and larger bubbles within the liquid solution. The solution passes through a gas separation component 134 to remove micro bubbles and larger bubbles, leaving a solution of gas nanobubbles within a liquid (AKA a nanobubble solution). Gas separated from the solution passes through an optional scrubber 136 before being released from the system (e.g., to atmosphere, to a house vacuum system). In some embodiments (not shown), the gas is reintroduced into the nanobubble generator to improve the efficiency of gas utilization. Nanobubble solution is passed through a delivery device 138 and delivered to a patient. In some embodiments (not shown), either the liquid solution or the nanobubble solution are reintroduced to the system before the pump. This recirculation of solution can increase the concentration of nanobubbles. In some embodiments, a nanobubble solution is warmed to a target temperature (e.g., body temperature) prior to exiting the system (not shown). In some embodiments, a nanobubble generation system includes a heater (e.g., electrical resistance heater) to warm a nanobubble solution.

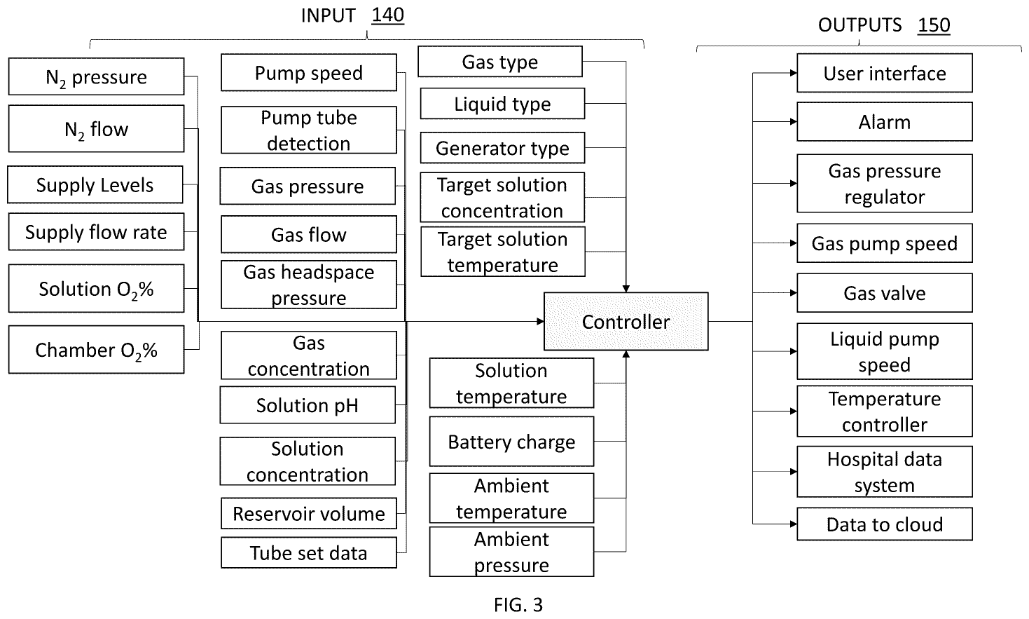

Any of the systems described herein can include a controller to control the generation of the nanobubbles, microbubbles, or any other type of gas-infused liquid solution. FIG. 3 depicts exemplary inputs and outputs of a nanobubble generation system controller. Inputs 140 include one or more of information about system components (e.g. pump speed, pump tube detection/insertion), information about the gas (e.g. gas type, gas flow rate, gas headspace pressure, gas supply pressure, gas concentration), solution concentration (e.g. target value, current value), information about the liquid (e.g. liquid type, a liquid volume measurement, a liquid mass measurement), tube set data (e.g. volume, expiration data, venturi size, etc.), system configuration information (e.g. generator type), battery charge level, and ambient conditions that could affect solution longevity (e.g. pressure, temperature). It will be understood that any data relating to the system, gas, liquid, and/or delivery device can be input into the controller. The controller generates outputs 150 that report system status to a user, generate alarms, modulate solution generation (e.g. gas pressure, gas flow rate, liquid flow rate, temperature), and report information (e.g. to a hospital data center, or to the internet cloud). Exemplary data to be reported to a hospital to the cloud include solution concentration, quantity generated, quantity administered, solution type, timing of delivery, duration of run time, system fault conditions, calibration status and other data. It will be understood that the controller can generate any output related to the system, gas, liquid, delivery device, nanobubbles, microbubbles, or other bubble types as needed.

### NO Solution

Nitric oxide nanobubble solutions are generated when NO gas is introduced to a liquid via a nanobubble generator. In some embodiments, the liquid is devoid of oxygen to prevent NO reaction with oxygen to form nitrite in solution. In some embodiments, the liquid includes dissolved oxygen which reacts with the nitric oxide to form nitrite. In some applications, the nitrite contributes to physiological effect such as vasodilation.

It can also be possible to convert nitrite back into NO in the solution. In some embodiments, potassium iodide can be used to convert nitrite back into NO. This results in safe amounts of iodine that would be injected into a patient. For example, it is understood that NO generation can be achieved by reducing nitrite ions with iodide ions in acidic conditions as follows:

2NO₂⁻+2I⁻+4H⁺→2NO+I₂+2H₂O

It is also understood that NO in oxygen-containing aqueous solution oxidizes mostly into nitrite as follows:

4NO+O₂+2H₂O→4NO₂+4H⁺

To eliminate nitrite in the NO nanobubble solution, stoichiometrically appropriate amounts of acid (such as HCl) and iodide (from KI) can be added to reduce nitrite back to NO.

Iodide can be toxic/harmful to humans at above 1.1 mg/day. In some embodiments, the injection volume can be controlled so that less than 1.1 mg of iodine gets injected into a patient per day. In some embodiments, iodine can be removed from the nanobubble solution through methods such as activated carbon or chlorination, as there may already be chloride ions in the solution from hydrochloric acid, or a combination of the two.

In some embodiments, vitamin C can be used to prevent nitrite formation. Vitamin C is a well-known strong antioxidant. Studies have shown that vitamin C not only can prevent the oxidation of nitric oxide to nitrite in aqueous solutions, but it can also reduce nitrite back to nitric oxide in slightly acidic conditions. The addition of vitamin C to the nanobubble solution (either before or after the generation of nanobubbles) could serve to prevent the formation of nitrite and/or keep the concentration of nitric oxide high.

In some embodiments, the oxygen level in the liquid only has to be low enough that when NO is added to the liquid, there will still be NO in the solution after all of the oxygen has reacted with NO. In other words, the amount of NO added to the solution can be more than the amount of oxygen in the solution in order to create a long lasting NO solution.

For the purposes of this document, it shall be understood that the term “NO solution” includes, at a minimum, a liquid and either dissolved and/or nanobubble NO.

### Oxygen Nanobubbles

Oxygen nanobubble solutions contain more oxygen per unit volume than dissolved oxygen alone. Oxygen is a necessary molecule for tissue health that can be utilized to treat anaerobic infections. A topically applied oxygen nanobubble solution can be used to treat affected tissue by 1) increasing oxygen content in tissues, making them healthier and more able to combat an infection, and/or 2) creating a high oxygen environment that is not compatible with anaerobic bacteria (i.e., anaerobes). Example clinical indications to treat with oxygen nanobubble solution include, but are not limited to, oral infections (e.g., infected gum pockets), skin infections (e.g., skin pores (acne), open wounds (i.e., avoid hyperbaric oxygen treatment), and hair loss. For example, in some applications, an oxygen nanobubble solution is delivered topically to the skin or gums. In some applications, an oxygen nanobubble solution is delivered via water flosser or oral irrigator.

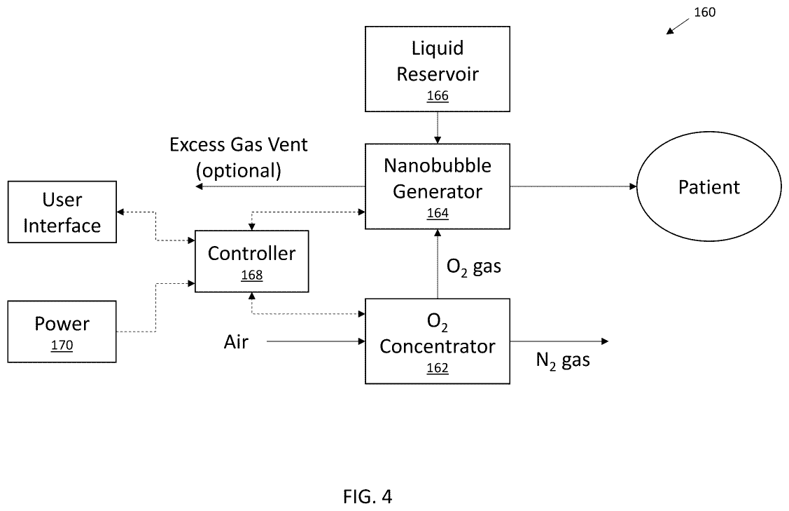

FIG. 4 depicts an exemplary embodiment of an oxygen nanobubble generation system 160 for patient treatment. Atmospheric air enters an oxygen concentration subsystem 162 (e.g., a pressure-swing adsorption device) that outputs an elevated oxygen gas flow (e.g., >21% oxygen, 90% oxygen) and a predominantly nitrogen gas flow. The elevated oxygen gas flow can enter a nanobubble generator 164 that creates a nanobubble solution from the oxygen-rich gas and a liquid sourced from a reservoir 166. In some embodiments, excess gas is optionally separated from the nanobubble solution and vented from the system to atmosphere. In some embodiments, excess gas is not separated from the solution and both solution and gas are delivered to the patient. Note that 95% oxygen dissolved as nanobubbles can be safer than using open oxygen gas flow in topical applications.

In some embodiments, the system is controlled by a controller 168 (e.g., a microprocessor) that receives input, for example, from a user interface (e.g., a button, a touchscreen, etc.). The controller 168 can be configured to manage the operation of the oxygen concentration and the nanobubble generator. In some embodiments, oxygen is separated from air and stored in a reservoir (not shown) before nanobubble generation. In some embodiments, oxygen separation and nanobubble generation occur simultaneously. The choice of when to make the oxygen and nanobubbles is related to one or more of available peak power, minimizing acoustic noise, nanobubble generation gas flow requirements, treatment solution flow requirements, amongst other considerations. Power is supplied from a power source 170, which can be an internal source (e.g., batteries) or an external source (e.g., AC/DC transformer from wall power). Delivery to the patient can be through any number of ways including but not limited to cannula, catheter, needle, topical application, and oral irrigator.

### Nanobubble Delivery

### Infusion

In some embodiments, a NO nanobubble solution is delivered through an infusion pump, a hand syringe, or IV set to a patient. In some embodiments, the delivery is intermittent (e.g., discrete boluses or a drip). In some embodiments, the delivery is continuous. Continuous delivery of NO solution to a location induces continuous vessel relaxation.

In some embodiments, a NO nanobubble solution is utilized to inhibit vasospasm. In some embodiments, a prophylactic dose of NO nanobubble solution is delivered as bolus (e.g., 75 ml up to 250 ml, depending on concentration). The size of the bolus may vary, for example, with patient size. In some embodiments, the NO solution is delivered in incremental steps over time (e.g., 5 ml every minute for 15 minutes). In some applications, the protective effect wears off over time (e.g., 0.5-2 hours), requiring additional solution to be delivered to maintain suppression of vasospasm.

In some embodiments, a continuous flow of NO solution is delivered to a patient intravenously over time before, during or after a medical procedure. In some embodiments, NO solution is delivered as a bolus. In some embodiments, timing and volume of bolus delivery is determined by either the clinician or the device controller based on one or more of primary treatment type, timing of primary treatment, patient body weight, and degree of existing patient vasoconstriction.

In some embodiments, the solution includes oxygen nanobubbles for oxygenating the treated tissue. In some embodiments, NO and oxygen nanobubbles are delivered simultaneously. In some embodiments, NO and oxygen nanobubble solutions are delivered in an alternating pattern. In an example, NO nanobubbles are delivered first to a constricted blood vessel to induce vasorelaxation, then oxygen-containing liquid (e.g., oxygen-containing blood, O₂ nanobubble solution, or dissolved O₂ solution) is delivered to the vessel to provide oxygen to the local and downstream tissues. In scenarios where vasoconstriction returns after a period of time (e.g. after the NO effect wears off), the process of delivering NO solution followed by O₂ solution can be repeated.

There are several metabolic pathways that NO can take when it is injected into the bloodstream in a nanobubble solution. Some NO rapidly combines with oxyhemoglobin to form methemoglobin. This is a rapid reaction, taking milliseconds, and can consume all available oxyhemoglobin in the immediate vicinity. Other NO travels within the blood stream as a nanobubble. Some NO converts to nitrite or nitrate within the blood stream, making it more stable and enabling it to travel greater distances within the body. Despite nitrite being less efficient at inducing vasodilation, when sufficient quantities are delivered, a therapeutic effect can be induced.

### Shelled Material

In some embodiments, nanobubbles are formed with a shell material, i.e., a material that encapsulates the gas using a low melting point polymer (˜37 deg C) for the nanobubble shell. The nanobubble shell can be formed from a variety of materials. In some embodiments, an oil is utilized to make the shell (e.g., palm oil, coconut oil, etc.). In some embodiments, a biocompatible wax is utilized to form the nanobubble shells (e.g., carnauba, bees). In some embodiments, the shell is comprised of a sugar (e.g., sucrose, dextrose, galactose, etc.). In some embodiments, a biodegradable polymer is utilized for the shell material (e.g., poly-lactic acid, polyglycolide, polycaprolactone, polyamide, polydioxanone, poly amino acids, polyanhydrides, poly (ortho) esters). In some embodiments, the diameter of the nanobubbles is in the range of 50 nm to 250 nm. Compared to the diameter of a red blood cell (typically 7.5 μm to 8.7 μm), the nanobubbles are quite small and do not present a risk of blocking capillary flow.

In some embodiments, a nanobubble solution system includes a mechanism to break the shell or otherwise promote the release of NO from the shell. In some embodiments, the shell is energized with ultrasonic energy (e.g., applied by an external applicator or ultrasonic catheter) to rupture the shelled bubble. In some embodiments, the nanobubble shell degrades or burst when exposed to blood chemistry and or a particular temperature (e.g., body temperature, 37° C.).

### Release

In some embodiments, NO solution is housed in a container that releases the solution in response to endogenous stimuli (e.g., pH, temperature, specific enzymes) or exogenous stimuli (e.g., ultrasonic excitation, acoustic sound waves, electric fields, magnetic fields, electromagnetic fields, or temperature). In some embodiments, the NO solution container is administered to a patient (e.g., injected, surgically, orally, nasally, intravaginally, intraurethrally, rectally, intraaurally, topically). In some embodiments, the NO solution is only released when specific endogenous and/or exogenous conditions are present. In one example, a NO solution capsule is swallowed by a patient and the capsule remains closed until the alkaline pH of bile acts upon it to breakdown the container seal and release the NO solution (e.g. an enteric coating). In another example, a NO solution capsule is intentionally left behind at a surgical site. The capsule is intermittently energized exogenously (e.g. transcutaneous RF energy, ultrasound) to release controlled amounts of NO solution to the surgical site to one or more of promote angiogenesis and treat/prevent infection. In another embodiment, NO solution is contained within a biodegradable container that releases NO solution after a period of time.

### Nitrate and Nitrite

In some embodiments, nitrate and/or nitrite are injected into the bloodstream to inhibit vasospasm. The nitrate reduces to nitrite over time. The nitrite reduces to NO over time (for example, 30 to 60 minutes). This approach provides a means to prolong the patient exposure to NO without the need for a continuous infusion. In some embodiments, a NO nanobubble solution is generated in a liquid that contains dissolved oxygen. The NO reacts with dissolved oxygen to form nitrite in the solution. In some embodiments, the level of dissolved oxygen in the liquid is controlled to ensure that there is NO remaining in solution after all of the oxygen has reacted with NO. Solutions containing one or more of NO, nitrite and nitrate are later injected into a patient.

### Clinical Applications

### Induced Hypoxia

In some embodiments, nitrogen nanobubbles in a liquid (e.g., saline) solution are introduced to a blood vessel. The nitrogen nanobubble solution is delivered locally to displace blood in the vicinity creating a region of hypoxemia. The hypoxic region of the vessel dilates. This effect can be utilized to facilitate thrombus removal, for example.

### Eye Treatment

In some embodiments, NO nanobubbles are introduced to a chamber within the eye to treat infection. In some embodiments, NO nanobubbles are introduced to the vitreous body of the eye to increase blood flow to the eye (e.g. increase blood flow through the retina). For example, NO nanobubble solution can be delivered to the eye to treat retinal vein occlusion.

### Kidney Protection

During cardiac surgery, thrombolytic materials (e.g., broken red blood cells) can be introduced to or formed in the blood stream. This material can draw endogenous NO from the endothelium of the vasculature, causing the vessel to contract. In some embodiments, IV infusion of NO nanobubbles into a blood stream during surgery can supplement endogenous NO levels and ensure that blood vessels remain patent. In one application, NO nanobubbles are delivered to the bloodstream before, during and/or after cardiac surgery to protect the kidney from damage related to loss of endogenous NO.

In some embodiments, a solution of NO nanobubbles is continuously infused into a patient for a treatment period. In some embodiments, the nanobubble solution is sourced from a reservoir. In some embodiments, the reservoir is part of a nanobubble recirculation loop that sources fresh fluid at roughly the same flow rate as the infusion rate. In some embodiments, a NO nanobubble solution is infused at one or more of before a surgical procedure, during a surgical procedure, and after a surgical procedure.

### Extracorporeal Membrane Oxygenation (ECMO) and Anticoagulation

In some embodiments, NO is utilized in ECMO to prevent coagulation of blood as it passes through the blood-gas exchange device. When NO is utilized, it decreases the need for heparin to be administered to prevent clotting. Hence, NO acts as an anti-clotting agent. In some embodiments, a NO nanobubble solution is delivered to a patient to prevent blood clotting during a procedure that presents a blood-clotting risk (e.g. ECMO).

### Vasospasm Inhibition

Vasospasm is a reaction of the blood vessel wall to a physical stimulus (e.g., contact, strain, pressure as can occur during intravascular procedures). It is a protective response by the vasculature that is triggered by nocicpetors (nerves that sense tissue damage) in the blood vessel wall. When the vessel is contacted (e.g., by a catheter, balloon, or arterial plaque), the vessel responds by contracting the smooth muscle in the vessel wall. This results in a significant decrease in blood vessel diameter and a many-fold decrease in cross-sectional area. Vasospasm presents risks to the heart due to an increase in flow resistance and to downstream tissue due to a significant decrease in blood flow.

Additionally, nitric oxide can inhibit the release of neurotransmitters involved in pain transmission, such as substance P.

NO is known to be part of the modulation of spinal and sensory nerve excitability. In some embodiments, introduction of NO to tissue can inhibit nitric oxide synthase (NOS) production.

In some embodiments, NO solution is administered for a period of time (hours, days, weeks) after intravascular surgery to prevent vasospasm.

### NO Delivery Via Balloon Catheter

In some embodiments, a balloon is inflated downstream from a spasm and inflated to a level that partially or fully obstructs blood flow. NO solution is introduced to the blood vessel to act upon the endothelium. NO in the solution reacts immediately with methemoglobin in the blood, however there can be more NO present than hemoglobin. In this case, NO interacts with the endothelium and dissolved oxygen in the blood stream. In some embodiments, NO solution is introduced to the blocked blood vessel at the balloon so that the solution flows in the retrograde direction to purge the stenotic region of blood. In some embodiments, the balloon is left in place for several minutes.

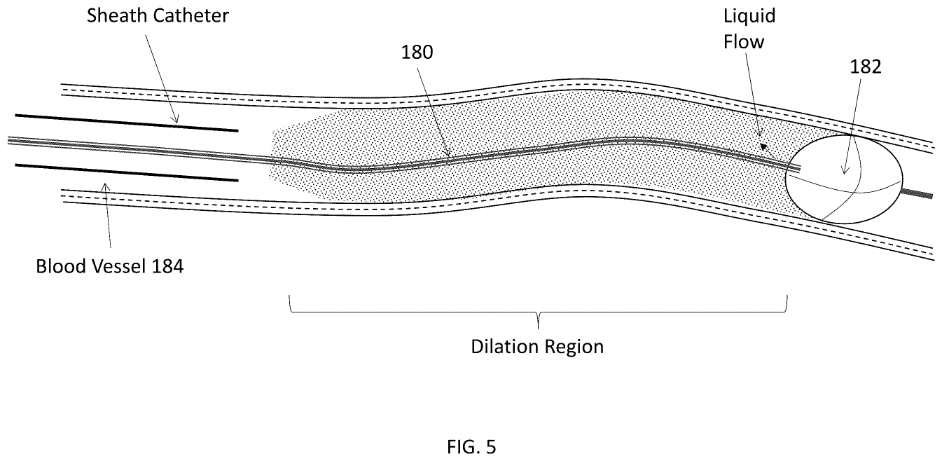

FIG. 5 depicts a NO nanobubble solution delivered via a balloon catheter 180. The catheter 180 is advanced to a point beyond a region to be dilated in a blood vessel 184. The balloon 182 is inflated by infusing a liquid or gas into the balloon thereby at least partially obstructing the blood vessel. FIG. 5 depicts complete flow obstruction. NO solution is introduced via a separate and independent lumen through an orifice near the balloon. The NO solution back-flushes the blood vessel to the nearest branch.

In some embodiment (not shown), a NO nanobubble solution is delivered to a first blood vessel (e.g., an aorta) in order to achieve an effect in a downstream vessel (e.g., an artery or arteriole). Although solution will be diluted by the blood flow and scavenged by hemoglobin, sufficient NO can still remain in solution downstream from the point of injection. In one exemplary experiment, the carotid artery in a rat was ligated. NO solution was introduced to the inferior carotid artery and flowed in a retrograde direction to the aortic arch where it entered the arterial flow. The effect of the NO solution on arteriole tension in the spinal trapezius muscle was measured using microscopic techniques and found to be significant.

### Secondary Drug Enhancement

In some embodiments, when NO solution is delivered in tandem with another systemic drug, it can have the effect of either prolonging the effect of the secondary drug or increasing the uptake of the secondary drug. In some embodiments, the secondary drug is one or more of a non-steroidal anti-inflammatory drug, and a pain medication (e.g. morphine).

### Urethra Treatment

In some embodiments, a patient with a bladder and/or urinary tract infection is treated with a NO nanobubble solution. In some embodiments, NO solution is introduced to the annular space between the wall of a catheter (e.g., Foley catheter) and the urethral wall. The NO in solution kills pathogens within the urethra as it flows through the urethra and out of the patient. In some embodiments, a urethral catheter includes a dedicated lumen for delivery of NO solution for lavage of the bladder and/or urethra. In some embodiments, NO solution is delivered to a bladder and/or urethra in the absence of infection (i.e., prophylactically) to prevent the onset of an infection.

### Gastric Treatment

In some embodiments, a NO solution is utilized in gastric lavage (e.g. to treat gastric ulcers, and or H-pylorus infections). In some embodiments, NO solution is delivered orally (e.g. drunk) to treat the lining of the gastrointestinal tract or increase NO within the patient.

### Infection Treatment

In some embodiments, a NO nanobubble solution is utilized to treat a microbial infection (e.g., protozoan, amoeba, bacterial, fungal, viral). Infection treatment may be delivered in a single bolus, as a continuous drip, and/or intermittently as discrete boluses administered at time intervals.

In some embodiments, a NO nanobubble solution includes a clotting agent (e.g., chitosan, thrombin, aprotinin, etc.) that promotes blood clotting when the solution is added to a wound. The clotting agent stops bleeding while the NO nanobubbles kill microbes that could otherwise infect the wound.

### Blood Transfusion

It has been proven that there is a reduction in coagulation during extracorporeal membrane oxygenation (ECMO) that if the oxygen sweeping gas includes NO, the blood is less likely to coagulate, and heparin is required to thin the blood. This same concept can be applied to blood transfusion whereby addition of a NO nanobubble solution to the blood can reduce the reliance on anticoagulants such as heparin.

### Raynaud's Disease

Raynaud's disease is a condition where the arteries of the hands and feet spasm in response to either cold temperature or stress. In some embodiments, a NO solution is applied to the skin of the affected body part (e.g., a hand, foot, nipple) that experiences poor blood circulation as can occur with Raynaud's disease. The NO passes through the skin and induces vasodilation in the underlying vasculature, thereby increasing blood flow to the treated region. In some embodiments, the NO-nanobubble containing material is in the form of a gel. Gels can be advantageous because their viscous properties promote adhesion to the skin and slow the loss of NO to the atmosphere.

### Wound Healing

In some applications, a NO nanobubble solution is applied topically to a wound to promote blood vessel dilation for improved oxygenation and to promote angiogenesis (i.e. the formation of new blood vessels). In some embodiments, a NO solution is applied to a diabetic ulcer. The NO within the solution disinfects the wound, induces vasodilation for improved perfusion and induces angiogenesis. In some embodiments, low pH NO solution (e.g. pH of 1.5-3) provides additional antimicrobial protection for the patient. In some embodiments, an antimicrobial solution is made with NO nanobubbles in vinegar.

### Dental Applications

NO solution can be applied to wounds within the mouth to promote healing and fight infection. In some embodiments, NO solution is applied to gum grafts, tooth sockets post-tooth extraction, and after dental surgery. In some embodiments, the NO solution is in the form of a gel (e.g. aqueous gel) that is less prone to migration from the target site.

### Topical Application

### Skin Applications

In some applications, NO solution is delivered through the skin using one or more (e.g., an array) of microneedles.

Skin, particularly oily skin as can be found on the face and in the ears, repels water, making the water roll off the skin surface. In some embodiments of a NO solution, a wetting agent that lowers the surface tension of the solution is added to reduce surface tension and improve contact with a target tissue (e.g., skin). Example wetting agents include but are not limited to propylene glycol, glycerin, diethylene glycol monoethyl ether, dimethyl sulfoxide, purified water, polyethylene glycol, alcohols (e.g., ethyl), petroleum distillates (e.g., polyacrylamides), sodium lauryl sulfate, quaternary ammonium compounds, and non-ionic surfactants (e.g. Triton X-100).

To enter through the skin, nanobubbles must first permeate the stratum corneum, the outermost layer of the skin consisting of dead cells, to reach viable cells. In some embodiments, a nanobubble solution includes ingredients that enhance the permeability of the stratum corneum to enable nanobubble transport. Sweat glands and hair follicles may also be utilized for solution transport, however they represent a small fraction of the entire skin surface area.

The layers of dead cells in the stratum corneum are bound together with lipids. NO is soluble in lipids. When there is a high concentration of NO external to the skin, there can be adequate diffusion gradient for NO to cross through the stratum corneum and into the subcutaneous layers. From there, the NO can enter blood vessels and/or the lymphatic system and be distributed systemically. In some embodiments, alcohols are utilized to increase fluidity of lipids in the stratum corneum which, in turn, facilitates nanobubble transport. In some embodiments, surfactants are utilized to solubilize the lipid components of the stratum corneum. This disrupts the wall of dead skin cells and opens channels for nanobubble transport.

Some embodiments of a NO nanobubble solution include a permeation enhancer (e.g., including terpenes, sulfoxides, laurocapram, pyrrolidines, fatty acids (e.g., mono-saturated, poly-saturated), fatty alcohols (i.e., alkanols), alcohols (e.g., glycol, propylene glycol, ethanol), surfactants, terpenes/terpenoids, and urea.

In some embodiments, the skin is exfoliated (e.g. roughened, abraded) prior to application of NO nanobubble solution to remove/thin the stratum corneum and decrease the resistance to transport of nanobubbles into viable tissues.

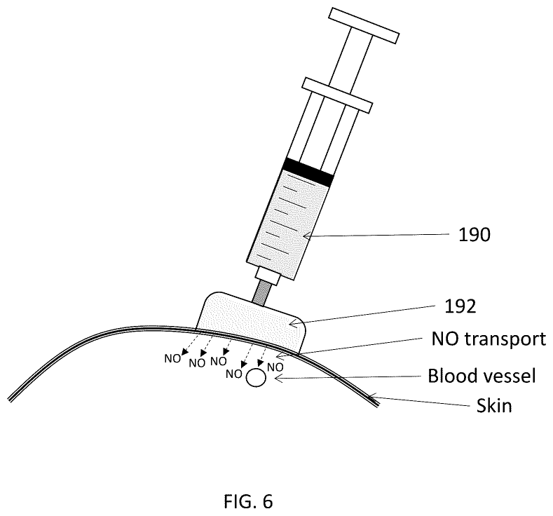

In some embodiments, permeation of a NO solution through a tissue barrier (e.g. skin) is promoted by a pressure gradient. In some embodiments, solution is pressurized against the skin with a device. FIG. 6 depicts an embodiment of a system for delivering nanobubbles that utilizes a syringe 190 to generate pressure within a pressure chamber 192 that is pressed against the skin. NO nanobubbles within the pressurized solution are transferred through the skin into underlying tissue. NO that enters blood vessels and lymphatic ducts can be distributed systemically.

In some embodiments, the skin is hydrated prior to NO solution delivery. Hydration of the stratum corneum facilitates transport of nanobubbles through the stratum corneum. In some embodiments, terpenes and terpenoids (terpenes with additional functional groups) are used to increase skin permeation of nanobubble compounds. In some embodiments, one or more of menthol, thymol, carvacrol, menthone, and cineole are utilized to improve skin permeation of lipid-based nanobubble solutions.

Fatty acids have been shown to improve permeability of skin. Examples of fatty acids include but are not limited to linoleic, alpha-linolenic, and arachidonic acids. In one specific example, oleic acid is used in some embodiments to mobilize and delipidize the stratum corneum.

In some embodiments, iontophoresis (i.e. the act of applying a low voltage to the skin with electrodes) is utilized to facilitate transport of NO nanobubbles across the stratum corneum layer of the skin.

### Frostbite

Frostbite is a condition when skin and subcutaneous tissues freeze. When tissue freezes, blood flow stops, and the tissue is at risk of permanent damage. Frostbite has traditionally been treated with gentle heat to thaw the tissue and restore blood flow. In severe cases of frostbite, tissue has been damaged directly by freezing (e.g. ice crystals and pierce cell walls and tissue structures) or indirectly by loss of blood flow. In some embodiments, affected tissue (e.g., foot, finger) is exposed (e.g., submerged, wetted, moistened, injected) to a NO nanobubble solution. The NO permeates tissue and dilates blood vessels to increase blood flow, thereby providing protection to tissue that has not been permanently damaged by being frozen.

### Subarachnoid Hemorrhage

Subarachnoid hemorrhage occurs when blood vessels within the brain leak blood into the cerebrospinal fluid. Over time (typically days), this condition can progress to a point that it affects blood vessel nerves within the brain, causing them to spasm. In one embodiment, NO solution is introduced intravascularly to inhibit vasospasm in a patient experiencing subarachnoid hemorrhage. In some embodiments, the NO solution is delivered as a periodic bolus (e.g., 75 ml every 1.5 hours). In some embodiments, the NO solution is delivered continuously (e.g., as an IV drip). In some embodiments, the NO solution is delivered continuously to the GI tract (e.g., through a nasogastric tube), where the NO is absorbed by the GI lining and enters the blood stream. In some embodiments, NO solution is delivered intrathecally (i.e., outside the vessel) to treat vasospasm.

Vasospasm can occur in response to multiple conditions including but not limited to aneurysm treatment (e.g., brain) whereby an aneurysm space is filled with coils of material, and mechanical thrombectomy. In some cases, NO solution is introduced to one or more of before, during and after the procedure to prevent or minimize vasospasm.

### Cerebrospinal Fluid (CSF)

In some applications, NO solution is introduced to the cerebrospinal fluid as NO has thrombolytic properties that can break clots that may form in the brain as the result of an aneurysm or subarachnoid hemorrhage. Blood vessel nerves are abluminal (i.e., on the outside of the lumen). Hence, nanobubbles in CSF can act on blood vessel nerves from the outside of the vessel to prevent vasospasm and/or reduce pain (i.e., nociceptor activity). In another embodiment, NO solution in CSF increases blood flow to the brain and/or spinal cord.

### Sexual Dysfunction

In some embodiments, a NO solution is utilized to treat female and/or male sexual dysfunction by dilating blood vessels to increase blood flow. In some embodiments, the NO solution is applied topically. In some embodiments, the NO solution is delivered via direct nanobubble infusion. In some embodiments, NO solution is administered as an adjunct therapy to existing hormone or systemic vasodilator therapy (e.g., estrogen and sildenafil).

### MRI Contrast

In some embodiments, polarized xenon gas is encapsulated in saline as a nanobubble. The solution is used as a contrast agent for MRI.

### Oxygen Delivery

In some instances, tissue with low oxygen content is treated with a hyperbaric chamber with elevated levels of oxygen. This approach can drive oxygen through tissue to improve tissue oxygenation, however working with high concentrations of oxygen presents safety risks. In some embodiments, tissue is treated with oxygen-containing nanobubble solution to improve tissue oxygenation.

### Lymphatic Treatment

The lymphatic system enables fluids within the body to be transported and equalize in pressure. The lymphatic system is a passive system with no blood present. Hence, there is little to no hemoglobin present within the lymphatic system. Owing to this lack of hemoglobin, the lymphatic system can be utilized to transport a NO nanobubble solution from one location of a body to another without risk of loss of NO to hemoglobin. Cancerous tumors can grow within the lymphatic ducts and nodes (i.e., the system). In one embodiment, cancer of the lymphatic system is treated by delivering a NO nanobubble solution to a cancerous tumor via a lymphatic duct.

### NO Nanobubble Solution Chemistry

### Oxygen Removal

NO reacts rapidly with oxygen to form nitrogen dioxide. When NO is added to a liquid to form a nanobubble solution, NO in the solution will react with any oxygen within the solution. In some embodiments, a NO nanobubble solution generation system receives oxygen-free liquid for generation of nanobubbles from an external source. In some embodiments, a NO nanobubble generation system receives oxygen-containing liquid and removes the oxygen prior to nanobubble generation.

Various methods can be utilized by a NO nanobubble generation system to remove oxygen from the liquid. In some embodiments, the liquid is one or more of sparged (i.e., bubbled with an inert gas), boiled, sonicated, or passed over a membrane that pulls gases out of solution under vacuum to remove oxygen from solution prior to introduction of NO. In some embodiments, NO solutions are created in an oxygen-free (i.e., anaerobic) environment (e.g., a glove box, or an oxygen-free chamber) that is filled with an inert gas (e.g., nitrogen, argon). Oxygen-free gas may be received by a NO nanobubble generation system from an external source (e.g., a compressed gas cylinder) or generated by the system (e.g., pressure-swing adsorption with air to create nitrogen gas).

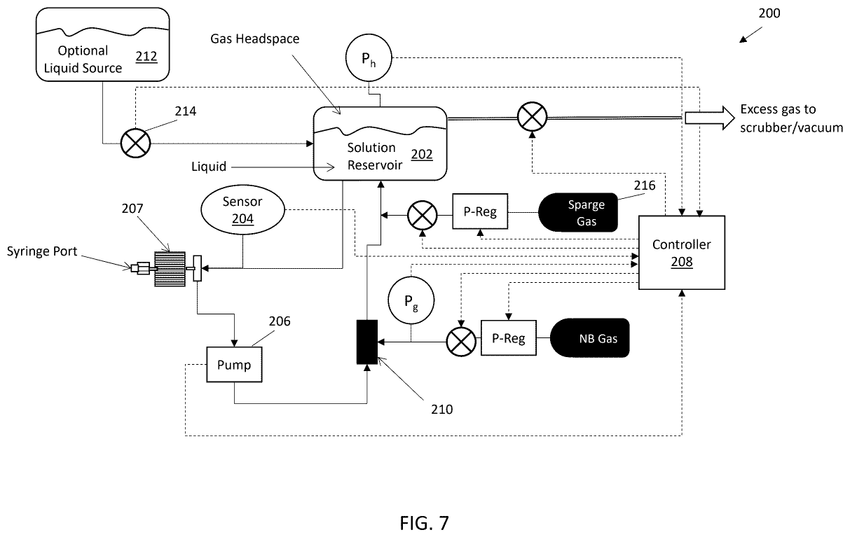

FIG. 7 depicts an exemplary embodiment of a nanobubble generation system 200 with sparging capability. Liquid is placed in a reservoir 202 with a gas headspace, and gas can be drawn from the bottom of the reservoir by a sensor 204 and outlet port, propelled by a pump 206. In some embodiments, an optional filter 207 before the pump removes particulate from the solution as it is collected. In some embodiments, the sensor measures dissolved gas within the liquid. In some embodiments, the sensor measures nanobubble size and/or quantity within the solution. In some embodiments, the controller 208 utilizes input from the gas sensor for one or more of identifying when the nanobubble (NB) gas supply has been exhausted, when the nanobubble quantity has reached a threshold value beyond which is acceptable to use.

Liquid leaving the pump enters a nanobubble generator 210 (e.g., a venturi assembly) where gas is introduced to the flowing liquid. Gas intended for nanobubbles flows from a pressurized source through a pressure regulator and flow control valve prior to the nanobubble generator. A gas pressure sensor located before the nanobubble generator is utilized by the controller to maintain target input conditions for the nanobubble generator and to identify when the gas supply has been exhausted.

After the nanobubble generator 210, the liquid solution includes nanobubbles, microbubbles, and larger bubbles. The solution returns to the reservoir 202 where bubbles larger than nanobubbles float to the surface and are released into the headspace. Gas within the headspace is release to a vacuum system or to atmosphere. In some embodiments, gas headspace release is passive and in other embodiments, the release is actively controlled via a flow controller (e.g., a valve), as shown. In some embodiments, gas headspace vents to a source of vacuum.

In some embodiments (not shown), gas in the headspace is passed through the nanobubble generator 210 a second time. In some embodiments, gas in the headspace is delivered to the nanobubble generator actively (e.g., using a pump). In some embodiments, gas in the headspace is delivered to the nanobubble generator passively. For example, in one embodiment liquid flow through a venturi draws gas into the liquid. In another example, pressure within the reservoir headspace pushes gas into the nanobubble generator.

In some embodiments (shown), an optional fresh liquid reservoir 212 provides liquid to the solution reservoir. An optional valve 214, controlled by the controller, can be utilized to introduce fresh liquid to the nanobubble generation circuit. In some embodiments, the nanobubble generation circuit is installed initially with no liquid in it and the controller controls the filling of the nanobubble solution reservoir. In some embodiments, fresh liquid is added to the recircuit (e.g., at the solution reservoir) to make up for lost solution from the system as a User withdraws solution out of the system. In some embodiments, a fresh liquid reservoir is sparged to remove oxygen before the liquid is introduced to the nanobubble generation circuit (not shown).

A liquid can have a finite capacity for nanobubbles. Hence, it is disadvantageous to generate nanobubbles during the sparging process. For this reason, in some embodiments, sparging is accomplished by introducing an inert gas (e.g., nitrogen) to the liquid through another means than through a nanobubble generator. In some embodiments, the sparge gas is bubbled into the solution. In other embodiments, the sparge gas is passed through the reservoir headspace, drawing dissolved oxygen from the liquid with a diffusion gradient. The system in FIG. 7 depicts sparge gas sourced from a sparge gas reservoir 216. In the depicted embodiment, sparge gas is flows from a pressurized vessel through a pressure regulator and a valve that controls sparge gas flow. The sparge gas is introduced to the circuit or liquid reservoir to draw oxygen from the liquid while forming a minimal quantity of nanobubbles.

In some embodiments (not shown), a liquid is agitated with ultrasonic energy (e.g., a sonicator operating at >20 kHz) to drive oxygen nanobubbles to the surface of the liquid.

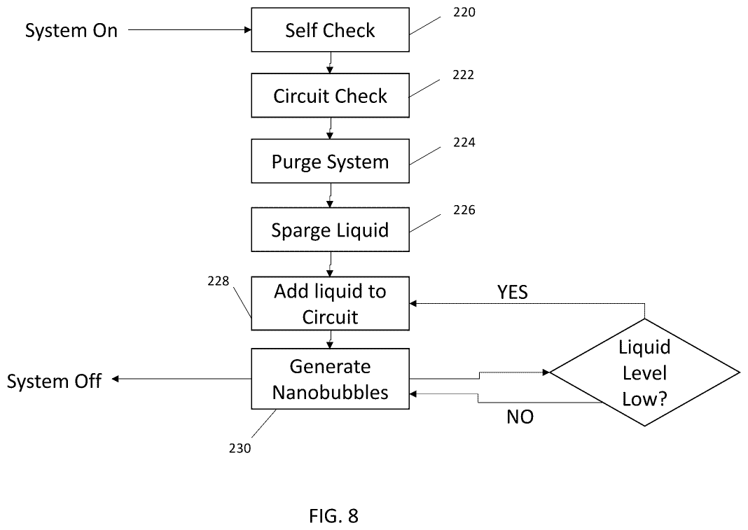

FIG. 8 depicts an exemplary control scheme for a nanobubble generation system. After the system is powered on, the controller completes a system self-check in step 220. The self-check includes one or more of checking that all sensor signals are within the expected range, pumps operate at the expected speed and direction (e.g., as indicated by encoders, flow and/or pressure sensors), and gas sources are present and at expected pressure. Upon successful completion of the system self-check mode, the system advances to circuit check mode.

In Check Circuit mode (step 222), the system confirms that the nanobubble solution circuit has been installed correctly. In some embodiments, the system confirms that the tubing has been installed into one or more pump heads (e.g., optically or with a displacement sensor), a liquid reservoir has been installed and has the expected mass (e.g., full of liquid, or empty depending on design), expiration date of the tubing set is within acceptable range, the tubing set has not been used previously.

In Purge mode (step 224), the controller opens one or more valves to permit a purge gas (e.g., nitrogen) to flow through the tube set and solution reservoir. This purging draws oxygen out of the components of the system. In some embodiments, purging is done when the tube set is devoid of liquid. In some embodiments, purging is done with liquid present so that oxygen is also removed from the liquid.

Next, the system enters a Sparge mode (step 226). In Sparge mode, purge gas is bubbled or passed over liquid to remove oxygen. In some embodiments, sparging is done in the liquid source reservoir. In some embodiments, sparging is done after liquid is added to the liquid circuit. In some embodiments, Purge and Sparge modes are combined.

After Purge and Sparge modes are complete, there is little to no oxygen within the liquid, reservoir and tube set. If liquid has not been added to the circuit yet, it is added to the circuit in an “Add Liquid to Circuit” step (step 228). In some embodiments, a fixed amount of liquid is added to the circuit. In other embodiments, a variable amount of liquid is added to the circuit to bring the quantity of liquid within the circuit up to a target level. The level of liquid in the circuit can be measured by the controller via one or more sensors. Example sensor methods to quantify liquid in the system include but are not limited to the following: measuring the mass of the liquid reservoir, optically locating the liquid surface, capacitively measuring the quantity of liquid, measuring pressure within the liquid reservoir, and other methods.

The system enters a Nanobubble Generation mode (step 230) once all other modes have been completed. Nanobubble generation involves passing liquid and NO gas through a nanobubble generator (e.g., a venturi) to create nanobubbles within the liquid. In some embodiments, the liquid travels through the nanobubble generator in a single pass prior to use. In other embodiments, the liquid passes through the nanobubble generator multiple times to increase the concentration of nanobubbles in solution. In some embodiments, the liquid always passes through nanobubble generator in the same direction, as with a recirculation loop. In some embodiments, the direction of the liquid reverses as liquid passes from one reservoir to another reservoir and back.

Nanobubble generation may be continuous or intermittent. In some embodiments, a system generates nanobubbles for several minutes and then pauses generation for a period of time. The period of time being related to a known nanobubble concentration decay rate. Once the quantity of nanobubbles in solution decreases below a certain threshold, nanobubble generation can automatically resume to maintain a target range of nanobubble concentration. In some embodiments, resumption of nanobubble generation is based on a period of time since the last generation period. In some embodiments, resumption of nanobubble generation is based on a measurement of nanobubble concentration within the solution (e.g., optically).

As the system generates nanobubbles, a user can draw a desired quantity of solution from the system. In some embodiments, the controller senses the quantity of solution in the system, as described above, and can automatically add additional liquid to the system from a liquid source to maintain a target volume of solution at all times.

### Oxygen Scavengers

There can be limits to how much oxygen can be removed from a liquid by degassing and inert atmospheres. Remaining oxygen gas can be removed from a solution chemically. In some embodiments, a NO nanobubble solution includes one or more of iron, acetic acid, sodium sulfite, sodium metabisulfite, sodium chloride, activated carbon, acetic acid, ascorbic acid, and citric acid to scavenge oxygen within the liquid. In some embodiments, oxygen scavenger materials are introduced to the solution in the form of a filing or a pellet. In some embodiments, the NO-containing vessel is constructed from (e.g., iron) or lined with (e.g., ascorbic acid) materials that scavenge oxygen. In some embodiments, the solution is filtered through a particle filter to remove oxygen scavenger particles either during flow within the generation system or during removal of solution from the system.

### Nitrite Reduction

Reaction of nitric oxide with oxygen yields nitrite. In some embodiments, sufficient NO is delivered to a solution in dissolved and/or nanobubble form that there is residual NO in solution after a portion of the NO in solution has reacted with all oxygen in solution to form nitrite. In some embodiments, a reducing agent (e.g., ascorbic acid, Vitamin E, potassium iodide) is added to solution (before, after or during NO addition) to reduce the nitrite to NO and dehydroascorbic acid. NO gas formed in this way is not within a nanobubble and either become dissolved within the liquid, enters the liquid vessel headspace, or enters the atmosphere (in cases that are open to atmosphere). Once all oxygen molecules have been removed from solution, a primary vector for NO loss has been eliminated and NO nanobubbles have significantly longer longevity in solution. For example, NO nanobubbles made in aerobic water will last roughly 30 minutes. NO nanobubbles in solution with no oxygen, as can be created with the use of a reducing agent, will remain potent for multiple days to weeks to months.

In some embodiments, nitrite (e.g., sodium nitrite) is added to the liquid before NO nanobubble production. The presence of nitrite in solution inhibits the formation of nitrite from nitric oxide per Le Chatelier's principle. This can inhibit the loss of NO from nanobubbles thereby maintaining a higher concentration of NO in solution. For applications that do not require nitrite, nitrite can be removed from the solution by passing the solution through a reducing scrubber (e.g., ascorbic acid). Nitrite removal can be done at the time of solution manufacture, at the time of solution packaging, or at the point of use.

The quantity of NO in solution is related to the concentration of NO in the initial gas, the duration of time that the NO is introduced to the liquid via the nanobubble generator, the efficiency of the nanobubble generator, the initial quantity of oxygen in the liquid, and/or the presence/absence of a reducing agent. Some reducing agents (e.g., ascorbic acid, potassium iodide solutions) require additional hydrogen ions available to promote the reduction reaction. Hence, pH that is lower than neutral (i.e., acidic environment) is required for the reduction reaction to take place. In some embodiments, an additional acid (e.g., sulphuric acid or HCl) are added to the solution to lower the pH and enable the nitrite reduction reaction to occur. Completion of the reaction of the reaction of nitric oxide with oxygen to form nitrite is limited by oxygen levels and can therefore be detected with a dissolved oxygen meter. In other words, the dissolved oxygen content level will be zero when all the oxygen has reacted with NO for form nitrite.

In some embodiments, the reducing agent is added to the solution directly and becomes part of the solution. In some embodiments, the solution is passed through a scrubber or matrix containing the reducing agent (i.e. a reducing scrubber). For example, the reducing agent can react with nitrite in the solution without entering the solution or minimally entering the solution. After the reducing steps are completed, the solution is removed from the NO generation system, leaving the reducing agent (or at least most of it) in the NO generation system. In some embodiments, NO solution is poured through a matrix of reducing agent (e.g. a granular ascorbic acid scrubber) within an oxygen-free environment. Liquid exiting the scrubber is (for the most part) devoid of nitrite and reducing agent.

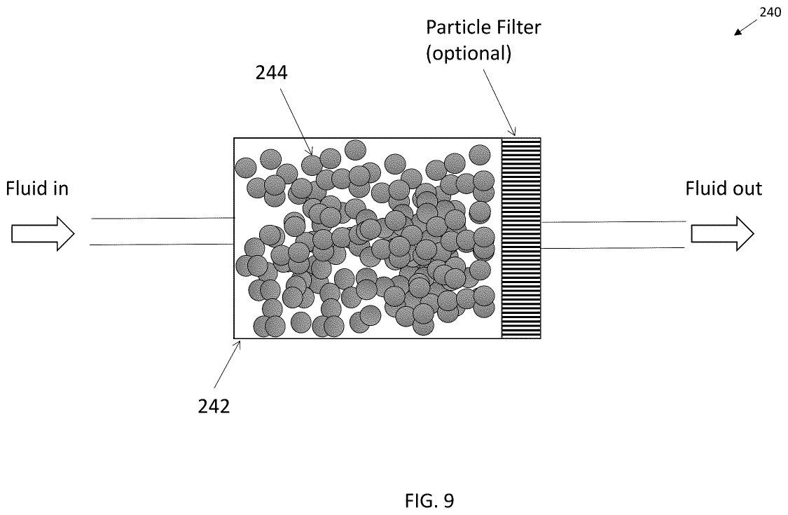

FIG. 9 depicts an exemplary embodiment of a scrubber 240 formed of particles of reducing material for removal of nitrite from a NO solution. The scrubber comprises a housing 242 that holds the reducing material or reducing agent particles 244. The reducing material is within a fluid path so that surface area of the reducing material is exposed to fluid that flows through the housing. In some embodiments, the reducing material is in the form of particles, spheres, or granules (shown). In some embodiments, the reducing material is in the form of fibers, amorphous material, a membrane, or a textile (not shown). In some embodiments, the reducing material is in the form of grooved sheet material (stacked or rolled into a spiral), or sheet material that is spaced apart with spacers to form channels for fluid flow (not shown). In some embodiments, the particles are 100% reducing material. In other embodiments, the particles consist of a substrate material that is coated with reducing material. Exemplary substrate materials include but are not limited to silica gel, zeolite, sodium alginate, alumina, glass, and polymer. In some embodiments, the particles are made of a composite material that includes, in-part, the reducing material. In some embodiments, the reducing material is chemically bound to a substrate material to prevent introduction of the reducing material into the NO solution. The particles reside in a chamber within the housing. NO solution enters through an inlet on the left side and exits through an outlet on the right side. An optional particle filter is included to capture particles from the reducing material and particles from other sources that are in the NO solution. In some embodiments, the particles are packed tightly within the scrubber. In some embodiments, the particles are loosely packed. In some embodiments, the orientation of the scrubber is vertical with respect to gravity, like a fluidized bed scrubber.

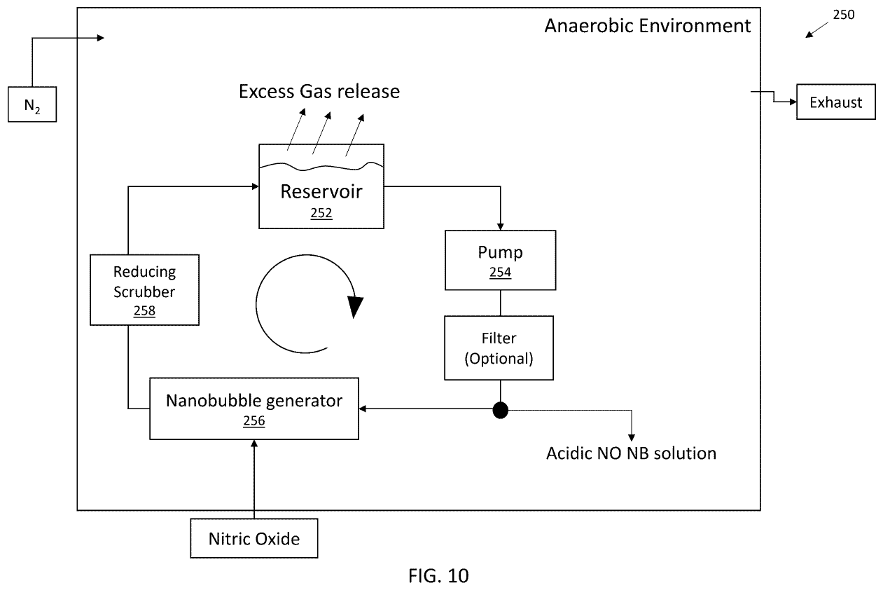

FIG. 10 depicts an exemplary embodiment of a NO nanobubble generation system 250 that utilizes a reducing scrubber. Liquid is sourced from a reservoir 252 and pushed through the system by a pump 254. The liquid passes through a nanobubble generator 256 (e.g., a venturi) where NO gas is introduced to the liquid flow. Gaseous and nanobubble NO reacts with any oxygen in the solution, forming nitrite. The liquid then passes through a reducing scrubber 258 that reacts with nitrite in solution to form additional NO. The liquid returns to the reservoir where gaseous NO is released into the anaerobic environment around the circuit. An optional particle filter can be placed anywhere along the flow path. When placed after the pump and before the fluid sample port, the filter captures particles from all parts of the system, including the pump, immediately prior to removal from the system. The liquid removed consists of NO nanobubbles in the substrate liquid.

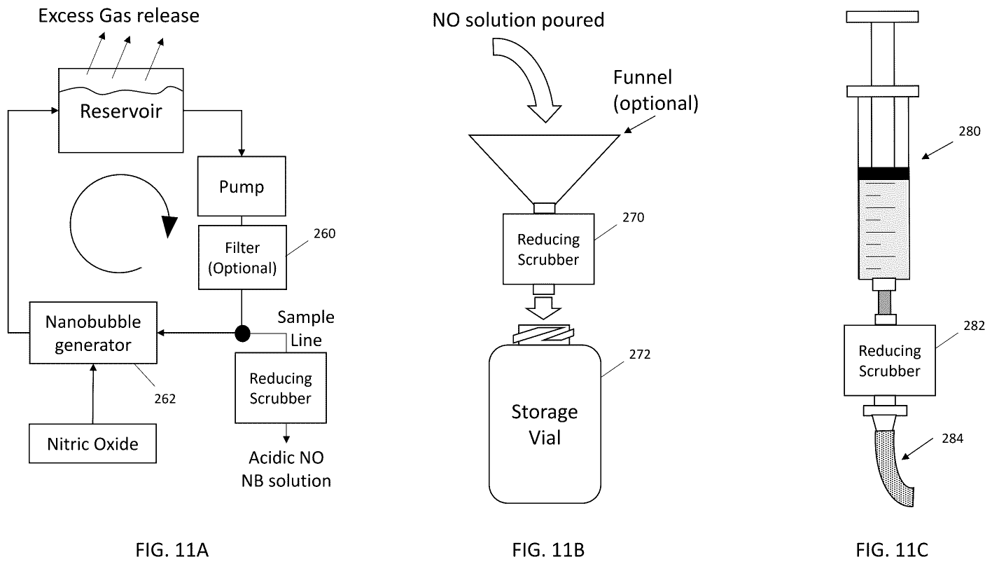

In some embodiments, a nanobubble solution is passed through one or more of a reducing scrubber and a particle filter to remove nitrite and particulate, respectively, after the solution is removed from the nanobubble generating system. FIG. 11A depicts an embodiment where the reducing scrubber 260 is part of the sample flow path on the nanobubble system (shown). As liquid exits the nanobubble solution generator 262, the liquid is one or more of scrubbed for nitrite and filtered for particulate.

FIG. 11B depicts an embodiment where a NO nanobubble solution is transferred (e.g., poured, pumped, etc.) through a stand-alone nitrite scrubber 270. As shown, the stand-alone reducing scrubber includes an optional funnel. In some embodiments, the standalone reducing scrubber also includes a connector to a storage vial 272. The NO solution is directed to a storage container (glass vial depicted) which is subsequently capped. is a separate device that the nanobubble solution is passed through as it flows into a container.

FIG. 11C depicts an embodiment where a NO nanobubble solution within a syringe 280 is pushed through a stand-alone reducing scrubber 282 as the NO nanobubble solution is introduced to a catheter 284. In some embodiments, the standalone reducing scrubber includes Luer fittings, to ensure secure and leak-free connections.

### Solution pH

After all oxygen has been removed from the solution in the form of nitrite, in some embodiments, the solution is left at a low pH with excess ascorbic acid to remove any nitrite that may form during solution storage as the results of oxygen molecules entering the packaging. Some applications such as antimicrobial infection treatments may benefit from low pH. Other applications may have limits to solution acidity. In applications where a low pH is not acceptable, the pH of a solution may be raised using one or more of an alkaline agent, a buffering agent after nitrite reduction, or dilution.

Solution neutralization can be done at the time of manufacture and prior to packaging or immediately prior to use. Neutralizing a NO solution immediately prior to use allows for the protection provided by low pH (i.e., reversal of nitrite formation) during storage. In some embodiments, the amount of neutralizing agent is predetermined and simply added to the solution. In some embodiments, the quantity of neutralizing agent is a variable amount, with the amount added depending on one or more of the target pH and response of the NO solution. In some embodiments, the solution mixing system includes a pH sensor. In some embodiments, the solution mixing system includes a controller that utilizes a pH measurement to vary the amount of neutralizing agent added to achieve a target pH level in solution.

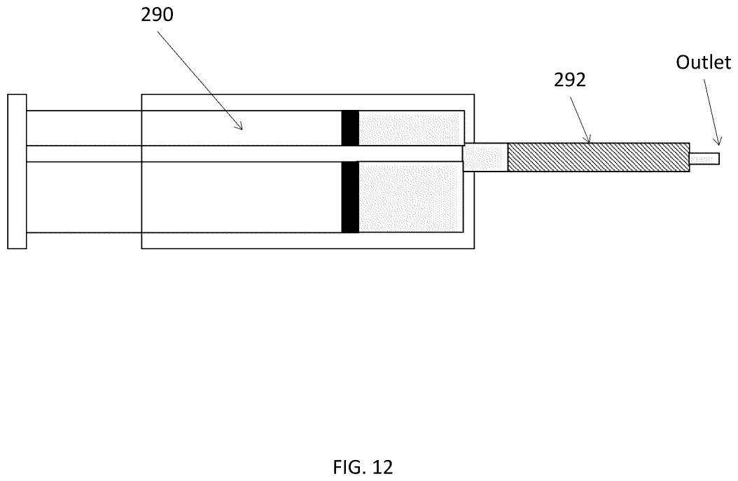

Various means can be utilized to neutralize a NO nanobubble solution prior to use. FIG. 12 depicts a dual syringe 290 with mixing tip. As the syringe is compressed, nanobubble solution in one chamber is mixed with a neutralizing solution prior reaching the outlet. An optional static mixer 292 can increase the level of mixing between the two solutions. In some embodiments (not shown), a syringe is first partially filled with neutralizing solution and then further filled with nanobubble solution so that the nanobubble solution is neutralized within the syringe prior to delivery to a patient.

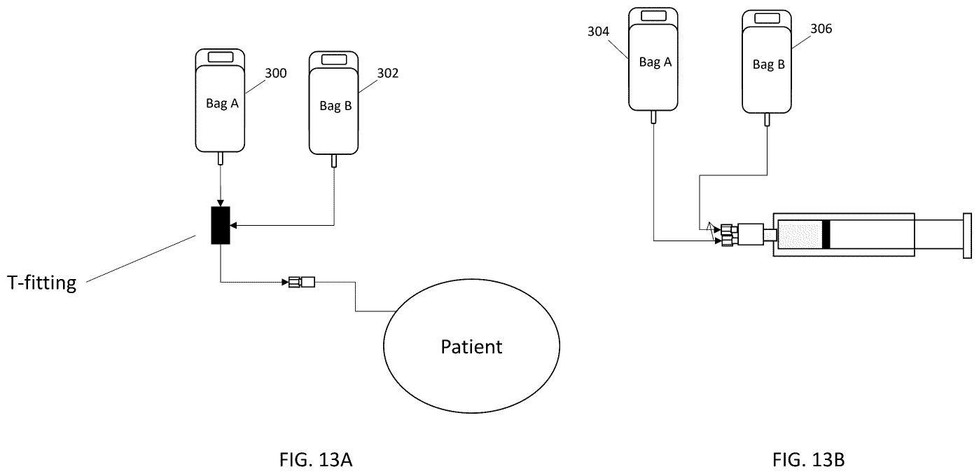

FIG. 13A depicts an embodiment of a system that includes NO solution in a first reservoir 300 (e.g., an IV bag) that is diluted with a buffering agent in a second reservoir 302 (e.g., an IV bag). The two bags are drained at appropriate flow rates to neutralize a NO solution to a target pH at the time of delivery to a patient thereby minimizing the potential for the NO solution to encounter oxygen before delivery.

FIG. 13B depicts an embodiment of a system that provides NO nanobubble solution and neutralizing material in separate reservoirs 304, 306 (e.g., IV bags). Each reservoir is connected to a check valve (e.g., a syringe-activated Luer fitting). When a syringe is connected, the syringe draws fluid from both reservoirs to that the NO solution and neutralizing solution mix within the syringe. In some embodiments (not shown), the NO solution is maintained at a different temperature than the neutralizing solution to prolong the life of the NO solution. When the two solutions mix, however, the temperature of the mixture is acceptable for treating a patient. In some embodiments (not shown), the syringe is filled in series with a first solution and then a second solution. Maximum NO retention is achieved when NO solution is introduced to the syringe last.

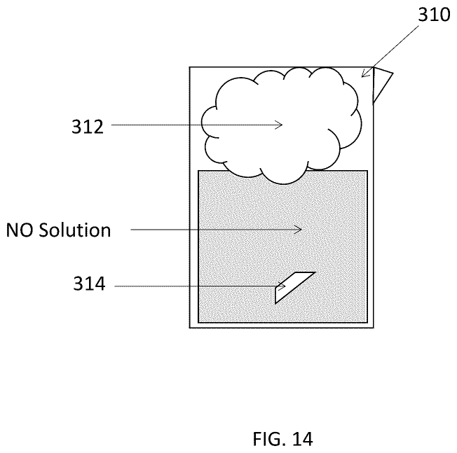

FIG. 14 depicts an exemplary embodiment of a reservoir or container or vessel 310 (e.g., a beaker) of NO solution with optional inert gas in the headspace 312 (e.g., argon). In some embodiments, the NO solution vessel has a neck or a tapered neck to increase headspace and decrease gas exchange with the environment. In some embodiments, a solid or powdered base 314 is added to the solution to neutralize prior to use. The base dissolves into the NO solution to raise the solution pH to neutral or near-neutral (e.g., 5 pH to 8 pH) prior to use. One advantage of utilizing a solid base is that it can have little to no oxygen content. In some embodiments, a liquid base is added to the solution to raise the pH. Ideal liquid bases have little to no dissolved oxygen. After neutralization, liquid is withdrawn from the container to a syringe for delivery to a patient. Syringes can be made from either polymer, metal, ceramic, or glass. Glass syringes have less dissolved oxygen.

### Solution Ions

In some embodiments, ions are present in a nanobubble solution to increase nanobubble stability (e.g. electrolyte solutions, saline). Ions can prolong a nanobubble solution by altering the nanobubble surface charge and enhancing repulsion between bubbles. The quantity of ions in solution can also affect the maximum concentration of nanobubbles that can be achieved in a particular liquid and gas combination for a nanobubble solution.

### Perivascular Delivery

In some applications, NO solution is delivered to the periphery of a blood vessel. In some embodiments, delivery is via needle or catheter to the abluminal space. In some embodiments, delivery is by an intravascular catheter that can cross the vessel wall. In some embodiments, an intravascular catheter includes a fluid delivery lumen that is in fluid communication with one or more articulating needles that can be deployed through the vessel wall.

### Exemplary NO Nanobubble Solution Procedure

In an exemplary embodiment, a NO nanobubble solution can be derived as follows: 300 ml of water (e.g., distilled water) is mixed with 1.5 ml of 1M sulfuric acid (or equivalent acid) and 1.5 g of ascorbic acid. The ascorbic acid is added to remove nitrite from the solution. The sulfuric acid is added to lower the pH to enable the ascorbic acid-nitrite reaction. In some embodiments, ascorbic acid is used alone and in sufficient quantities to lower the solution pH to an effective level. The solution is passed through a nanobubble generator (e.g., a venturi or any nanobubble generator) one or more times to generate nanobubbles in solution. Flow through the nanobubble generator is propelled by a pump (e.g., a diaphragm pump). The optimal flow rate of liquid through the nanobubble generation depends on the type and size of the nanobubble generator. In some embodiments, this optimal flow rate is in the range of 1 to 10 1 pm. It will be understood that the amounts of each part of the nanobubble solution can vary.

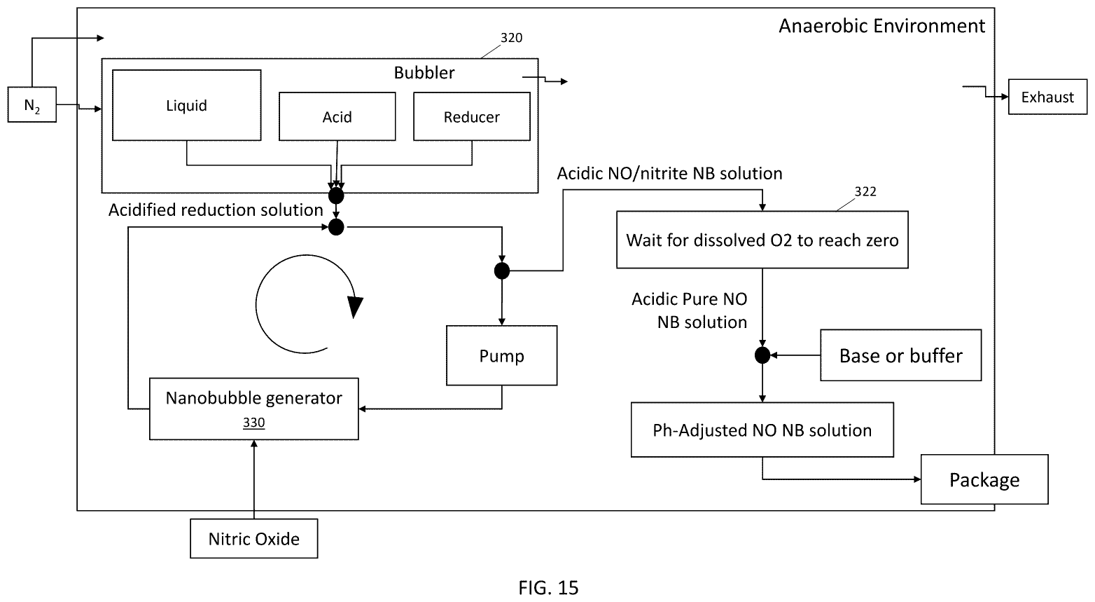

FIG. 15 depicts an exemplary process for generating a nanobubble solution. In some embodiments, the entire process takes place in an oxygen-free environment (labeled “Anaerobic Environment”). First, a liquid is combined with an acid (e.g., HCl, Sulfuric acid) and a reducer (e.g., Vitamin C, Vitamin E, etc.) in step 320 to form an acidified reduction solution. In some embodiments, the initial liquid is deionized water. The solution is then purged of oxygen (step 322) by bubbling an inert gas (e.g., N₂ sparging shown) to form deoxygenated acidified reduction solution (DARS).

In some embodiments, the deoxygenated liquid is deoxygenated as part of the process (shown) (e.g., bubbling with nitrogen gas for a period of time), In some embodiments, the liquid is sourced in a deoxygenated state (not shown). For these purposes, “Deoxygenated” does not mean completely devoid of oxygen but instead, “Deoxygenated” means that oxygen levels are either naturally low (i.e., oxygen is not very soluble in the selected liquid) or have been reduced to low levels with the understanding that any remaining oxygen will combine with NO in the solution to form nitrite. In one exemplary embodiment, dissolved O₂ levels are reduced to 0.1 ppm during the deoxygenation process.

In some embodiments, the DARS can be introduced to a nanobubble generation system. In some embodiments, the DARS is circulated within a nanobubble generation loop in which a pump propels the solution through a nanobubble generator 330 where NO gas is introduced to the liquid. In some embodiments, excess NO gas (i.e., gas that did not enter nanobubbles) is vented from the system or collected in a separate reservoir (not shown). In some embodiments (not shown), excess gas is collected from a headspace and is pumped back through the nanobubble generator again. Some embodiments are a single-pass system that do not include a recirculation loop, however that tends to result in a lower nanobubble concentration in solution. Maximum nanobubble concentration is achieved by passing the solution through the nanobubble generator multiple times. Further optimization can be achieved by minimizing vibration imparted to the solution (e.g., pump vibrations) and selecting flow rates and tubing sizes that maintain laminar solution flow.

In some embodiments, additional DARS is added to the recirculation loop as needed, to make up for lost solution to other parts of the system. Within the nanobubble generator, NO-containing gas is introduced to the DARS. Each NO molecule follows one of the following pathways:
1) Form NO-containing nanobubbles in solution,
2) Dissolve into solution,
3) React with oxygen in solution to form nitrite, or
4) Form larger bubbles that float out of solution and are released from the system.

After NO loading, the solution because an acidic NO/nitrite solution. The acidic NO/nitrite solution is drawn from the recirculation loop either actively (e.g., by pump) or passively (e.g., by gravity) and allowed to age sufficient time that all or nearly all O₂ has reacted with NO to form nitrite and all nitrite has been reduced. Measurements of 0.00 ppm O₂ by mass have been measured in solutions following this process. In some embodiments, this aging process occurs within the recirculation loop when the solution circulates within the loop for a sufficient duration of time. With no remaining nitrite in solution, the solution is referred to as “Acidic Pure NO nanobubble solution.”

In some embodiments, once the final solution pH has been established, the solution can be poured into vials within the anaerobic environment. Glass vials can be used since they are impermeable to oxygen. Typically, the vial is filled completely to minimize any gaseous headspace between the solution and the vial cap. In some embodiments, the headspace is filled with inert gas (e.g., nitrogen, argon, etc.) The vials are then placed in pouches with an oxygen scavenger and the pouches are heat sealed prior to removal from the anaerobic environment.

In some embodiments, where a higher pH is desired for the solution application, a base (e.g., NaOH, Ca(OH)2, KOH, etc.) and/or buffer (e.g., Tris, Phosphate, etc.) is added to the solution. An increase in pH can disable the ability of the reducing agent (e.g., ascorbic acid) from protecting the solution from oxygen. In some embodiments, the pH of the solution is shifted towards neutral prior to packaging of the solution. In some embodiments, the solution is packaged in an acidic state and neutralized prior to use, which is typically in an aerobic environment.

In some embodiments, an acid can be used in the DARS that has a conjugate base. For example, sulfate has slight vasodilatory properties, and the chloride from hydrochloric acid may help balance charges on the nanobubble surface. Ascorbic acid can then be added to the acidified liquid. Due to the low magnitude of NO concentration in the solution, ascorbic acid does not need to be of high concentration in the solution (far below human body's pH tolerance for an injection).

### Nitrite Scrubber

The reaction between nitrite and reducing agents can be rapid, enabling real time scrubbing of nitrite from a solution. In some applications, a NO-containing solution is delivered through a bed of reducing beads (e.g., ascorbic-acid coated beads) to a patient, thereby eliminating delivery of nitrite. In one embodiment, where some NO is lost from a solution during storage due to oxygen entry into packaging, the limited amount of nitrite formed by the oxygen can be removed prior to use of the solution. In some embodiments, the reduction scrubber can be part of a syringe or an accessory to a syringe.

### Buffered Solution

In some embodiments, an alkaline liquid is utilized for nanobubble generation. The alkaline properties of the liquid neutralize acid that can be formed when NO reacts with gaseous oxygen to form NO₂ and subsequently nitric acid.

### Surfactants

In some embodiments, the liquid utilized in nanobubble generation includes one or more surfactants. The surfactants promote bubble formation. Examples of surfactants utilized in nanobubble generation include but are not limited to Tween 80 and polyvinyl alcohol (PVA).

### NO Solution Packaging

In some embodiments, a NO solution is packaged in a first container (e.g., a jar, bag, syringe, vial, etc.). The first container may be made from a chemically inert material such as polymer, ceramic, metal or glass. The first container is then placed in a second container along with oxygen scavenging material. Oxygen that enters the second/outer container is captured by the oxygen scavenging material and does not enter the NO solution within the first container. In some embodiments, a sealed mylar pouch is utilized as the second container. In some embodiments, packaging materials are soaked in an anaerobic environment for a period of time before use to minimize oxygen loading. In some embodiments, packaging materials are heated and/or physically scrubbed to enhance oxygen removal from surfaces prior to use.

In some embodiments, gases within the nanobubble generation and or packaging system are pressurized to prevent atmospheric oxygen from entering the system and associated nanobubble solutions. In one example, an oxygen-free gas within the secondary container (e.g., a pouch or cannister) is pressurized to prevent atmospheric air from entering the container. In another example, a flexible NO solution reservoir (e.g., an IV bag) is squeezed (e.g., with a pressure cuff or a clamp) to maintain pressure within the solution and gas headspace to prevent atmospheric oxygen from entering the system. A soft-walled NO solution container can be used as the volume of the vessel decreases as NO solution exits the container thereby preventing other materials (e.g., oxygen-containing gases or liquids) from entering the vessel after NO leaves, thereby protecting the solution from reacting with oxygen.

The container wall thickness contributes to oxygen permeability with thicker walled materials providing greater protection from oxygen. Material selection also is important with some materials (e.g., metals, ceramics, glass) having less oxygen permeability than other materials (e.g., polymers). Polymers with higher resistance to oxygen transport typically have high crystallinity and low free volume. In some embodiments, one or more of polyvinylidene chloride, ethylene vinyl alcohol copolymer, polyvinyl alcohol, methylcellulose, and hydroxypropyl methylcellulose-based materials are utilized to package NO solutions due to their low oxygen-permeability. In some embodiments, a NO solution is packaged within one or more protein-based films which block oxygen transport due to their polarity and linear (non-ring) structure.

In some embodiments, the nanobubble generation circuit is constructed entirely of rigid materials. Propulsion of fluid through the circuit can be accomplished via a gear pump, piston pump or other type of pump with rigid components. In some embodiments, the nanobubble generation circuit is constructed entirely of rigid materials except for the pumping mechanism (e.g., a polymer diaphragm in a diaphragm pump or a polymer tube placed in a peristaltic pump).

In some embodiments, NO solution is kept within a soft-walled container (e.g., a bladder) with a valve or cap. This type of container provides an advantage in that the volume of the container can change. Hence, when NO solution is removed, the volume of the container is reduced, and oxygen-containing air does not enter the container.

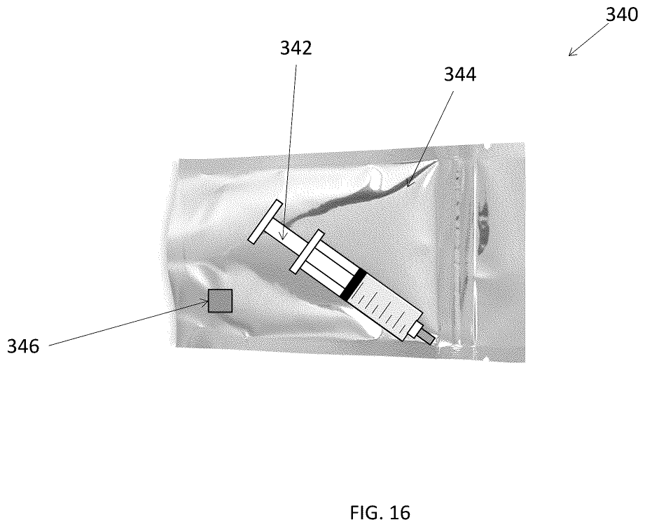

FIG. 16 depicts an exemplary packaging scheme 340 for a NO solution. NO solution is contained within a solution container 342 (e.g., a syringe) that is capped. The syringe is placed in a gas-impermeable container 344 (e.g., a mylar pouch) along with an oxygen scavenger material 346. The pouch sealed one or more times to prevent air from entering the pouch.

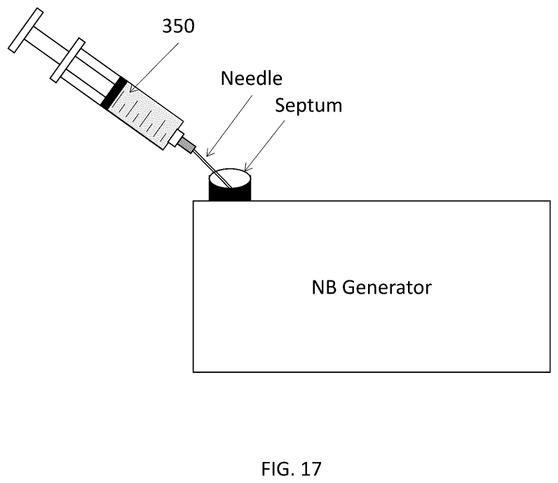

FIG. 17 depicts another embodiment for packaging a NO solution. NO gas is stored in a first container and liquid is stored in a second container. NO is introduced to the second container at the time that nanobubble solution is generated. In some embodiments, the first container is a syringe 350. In some embodiments, the first container is a compressed gas cylinder.

A nanobubble generation tube set includes a septum for introduction and extraction of liquids and gases. Prior to nanobubble generation, a needle is connected to an NO-filled syringe and used to pierce a septum in a liquid container. The NO is introduced to the liquid container through the septum. In some embodiments, the liquid container is devoid of any other gas (i.e., filled with deoxygenated liquid). Once the NO and liquid are in the same container, a nanobubble solution is generated (e.g., by microwave cavitation or by recirculating through a venturi). When the nanobubble solution has been generated, the same syringe can be utilized to withdraw nanobubble and transfer the solution to a clinical application.

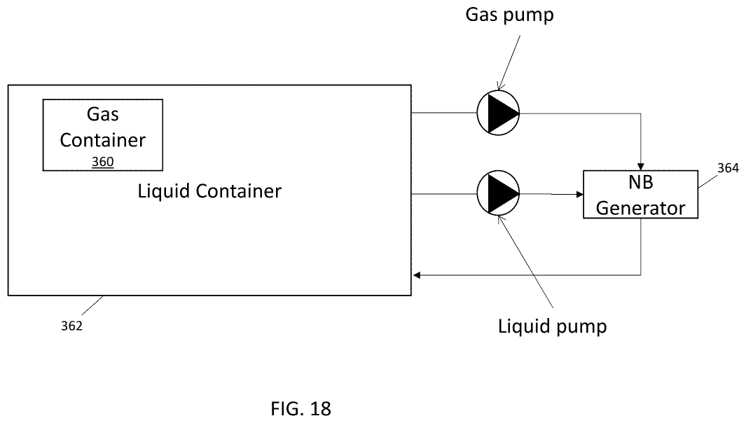

FIG. 18 depicts another embodiment of a packaging solution for a NO nanobubble solution. A first container 360 containing NO gas resides within a second container 362 containing a liquid during storage. Prior to nanobubble generation, the NO container is one or more of opened, ruptured, and punctured to permit NO to exit the container. Once the NO and liquid are in direct contact, a nanobubble generation device 364 generates nanobubbles of NO within the liquid. In the depicted embodiment, gas from the headspace is pumped into a nanobubble generator (e.g., a venturi) that generates nanobubbles within a flow of liquid from the liquid container. In some embodiments, the liquid container includes tubing for the transport of gas and liquid. In some embodiments, peristaltic pumps are utilized to prevent exposure of the nanobubble solution to air.

In some embodiments, when a nanobubble solution container is opened to atmosphere, atmospheric oxygen enters the container and dissolves into the solution, thereby beginning the decline of solution potency as NO in solution reacts with oxygen. In some embodiments, oxygen entry into the container is significantly reduced and/or eliminated by storing the nanobubble solution with a heavy, inert gas (e.g., argon) in the headspace. When the container is opened in a vertical or near-vertical orientation, the heavy inert gas, being heavier than air, remains in the container, settled at the surface of the nanobubble solution. Solution can be withdrawn by inserting a tube or needle through the argon layer to withdraw a quantity of nanobubble solution. Prior to recapping the solution container, additional heavy inert gas is optionally added to the headspace to displace any air that has entered the headspace. In one embodiment, the inert gas is sourced from a compressed gas cylinder.

Even with procedures in place to protect a NO nanobubble solution from oxygen, oxygen can still find its way into solution over time. Hence, it can be necessary to measure the potency of a NO nanobubble solution prior to use. In some embodiments, NO solution drawn from a storage container is tested for potency prior to use. In some embodiments, an in-solution NO analyzer is utilized to quantify the concentration of NO in solution. In another embodiment, NO solution is combined with one or more other materials to create a reaction. The presence and strength of the reaction is indicative of NO solution potency. In some embodiments, a sample of the NO solution is agitated to release NO gas. The NO gas then reacts with ozone, releasing photons, as done in chemiluminescent NO detection.

In some embodiments, NO solution is added to a container housing chlorine gas and agitated to release NO and form nitrosyl chloride, a yellow gas. When the quantity of reagents is controlled, the intensity of the yellow color of the nitrosyl chloride gas is indicative of the concentration of NO in solution. In another embodiment, NO solution is combined with fluorine gas to form an aqueous solution containing nitrosyl fluoride. When the nitrosyl fluoride solution is exposed to a metal (e.g., a thin strip of aluminum), the metal dissolves into its metal fluoride, releasing NO gas. In one embodiment, NO solution is introduced to a container containing fluorine gas with a metal strip. When potent NO solution is added to the container, the NO reacts with fluorine to form nitrosyl fluoride, which in turn dissolves the metal strip. The rate of dissolution of the metal strip can be an indicator of NO solution potency. If the strip does not dissolve, it is indicative that the NO solution had little to no NO present.

In some embodiments, NO solution is agitated to release NO into the headspace of a vessel, where it reacts with oxygen to form nitrogen dioxide, a brown gas. The density of the color of the brown color can be indicative of how much NO was in solution. In some embodiments, NO₂ gas is reacted with a solution of potassium bromide to form an electrical current and bromine in solution. Alternatively, NO₂ gas can be reacted with a solution of potassium iodide to generate iodide in solution and an electrical current. The electrical current can be indicative of the quantity of NO what was originally in solution.

In some embodiments, rather than testing for the presence of NO, the NO solution is tested for the byproducts of NO reacting with oxygen, specifically nitrite. By knowing the initial concentration of NO in solution, the concentration of nitrite in solution after complete NO oxidation can be calculated. When the calculated maximum nitrite concentration is utilized as a reference point and is compared to a current nitrite measurement, the ratio of the two nitrite concentrations provides an indication of NO solution potency. It should be noted that solutions that contain a reducing agent that requires an acidic environment may require neutralizing the pH prior to nitrite measurement. Without a more neutral pH, the reducing agent will continue to convert nitrite back into NO, thereby leaving no nitrite to measure.

In some embodiments, 4,5-Diaminofluorescein diacetate (DAF-2 DA), a fluorescent indicator, is added to the NO solution. The fluorescence of the solution is then measured with the quantity of fluorescence being indicative of quantity of NO in solution.

In some embodiments, the quantity of NO in solution is determined by electroanalysis. NO solution is added to a container so that it contacts one or more electrodes (e.g., carbon, platinum, etc.). A voltage is applied to the electrode and a current occurs as the NO in solution is oxidized on the surface of the electrode. High concentrations of NO in solution result in higher electrical current. In some embodiments, the electrodes are shielded with a perm-selective membrane that only permits NO to reach the electrode for greater sensor selectivity.

All patents, patent applications, and published references cited herein are hereby incorporated by reference in their entirety. It will be appreciated that several of the above-disclosed and other features and functions, or alternatives thereof, may be desirably combined into many other different systems or application. Various alternatives, modifications, variations, or improvements therein may be subsequently made by those skilled in the art.

1. A system for generating a medicinal solution, comprising:
a reservoir configured to store a liquid;
a source of medicinal gas;
a pump configured to propel the liquid from the reservoir;
a nanobubble generator in fluid communication with the reservoir and the source of medicinal gas, the nanobubble generator configured to form a nanobubble solution comprising nanobubbles of the medicinal gas in the liquid; and
a source of a reducing agent configured to be added to nanobubble solution to reduce nitrite to NO and dehydroascorbic acid in the nanobubble solution.

2. The system of claim 1, wherein the reducing agent is ascorbic acid, Vitamin E, or potassium iodide.

3. A nanobubble solution, comprising:
a liquid medium;
a medicinal gas in the form of nitric oxide (NO); and
a reducing agent configured to reduce nitrite to NO and dehydroascorbic acid in the nanobubble solution.

4. The nanobubble solution of claim 3, further comprising an alkaline agent to neutralize the nanobubble solution before administration to a patient.

5. The nanobubble solution of claim 3, wherein the nanobubble solution is used to treat vasospasm, subarachnoid hemorrhage, Raynaud's disease, sexual disfunction, infections, or frostbite.
